(19) United States
(12) Patent Application Publication
(10) Pub. No.: US 2006/0129035 A1
(43) Pub. Date: Jun. 15, 2006

(54) Devices, methods, and systems for measuring analytes extracted through skin

(76) Inventors: Kaoru Asano, Kobe-shi (JP); Takeshi Mizuno, Tsuchiura-city (JP); Yasunori Maekawa, Kobe-shi (JP); Kenichi Sawa, Amagasaki-shi (JP)

Correspondence Address: Tadashi Horie;Brinks Hofer Gilson & Lione, P.O. Box 10395, Chicago, IL, 60610, US

(21) Appl. No.: 10/745,443

(22) Filed: Dec. 23, 2003

(30) Foreign Application Priority Data
Dec. 27, 2002 (JP) 2002-381215
Mar. 27, 2003 (JP) 2003-089041
Mar. 27, 2003 (JP) 2003-089042

(51) Int. Cl. A61B 5/00

(52) U.S. Cl. 600/300

(57) ABSTRACT

Measuring devices for measuring an analyte extracted through skin of a living body are described that include: a noninvasive sampling unit for extracting the analyte from the living body through analyte transmission paths formed in an extracting region of the skin; an analyte detection unit for detecting a quantity of extracted analyte; and a control unit for obtaining a corrected analyte quantity based on the quantity of the extracted analyte and number of analyte transmission paths in the extracting region. Methods and systems for measuring analytes extracted through skin are also described.

### RELATED APPLICATIONS

The present application claims priority under 35 U.S.C. § 119 to Japanese Patent Application No. 2002-381215 filed Dec. 27, 2002, Japanese Patent Application No. 2003-39041 filed Mar. 27, 2003, and Japanese Patent Application No. 2003-39042 filed Mar. 27, 2003, the entire contents of which are incorporated herein by reference.

### FIELD OF THE INVENTION

The present invention relates to biological information measuring devices, measuring systems and measuring methods and, more particularly, to measuring devices, systems, and methods for percutaneously extracting analytes from a living body using noninvasive or minimally invasive extraction techniques, and for measuring the extracted sample with excellent reproducibility.

### BACKGROUND

The presence and amount of substances in a collected blood sample are generally determined in a clinical examination. Diabetics frequently measure blood sugar level, determine the dosage of insulin to administer based on the calculated blood sugar value, and perform self management of blood sugar levels to determine dietary restrictions and amount of exercise. Therefore, diabetics must measure their blood sugar level several times each day. Normally, measurement of blood sugar level is accomplished by collecting and measuring a blood sample using a puncturing tool or the like, causing a good deal of physical pain and burden to the patient. From this perspective, a simple examination that does not require blood collection and that is not burdensome to the patient would be strongly desirable.

In response to this desire, methods have been developed for measuring the amount and concentration of analytes noninvasively extracted from a living body without collecting blood. Known examples of such measuring methods include methods for percutaneously extracting an analyte by administering electric energy to the skin, such as the reverse iontophoresis method (e.g., U.S. Pat. No. 5,279,543; WO96/00110); methods for percutaneously extracting an analyte by reducing the barrier function of the skin and promoting passive diffusion by administering ultrasonic waves to the skin, such as the sonophoresis method (e.g., WO97/30628; WO97/30749); methods for percutaneously extracting an analyte by administering an enhancer to the skin, such as the chemical enhancer method; and methods for percutaneously extracting an analyte by applying negative pressure and suctioning the skin, as in aspiration methods (e.g., WO97/30628) and the like.

However, in the measuring methods and devices using these noninvasive extraction methods, the amount of extracted analyte changes over time, such that it is difficult to stably measure a quantity of an analyte. For example, the Glucowatch commercially marketed by Cygnus, Incorporated, must be worn for three hours prior to actually starting blood sugar measurement in order for the measurement to attain a state of equilibrium.

### SUMMARY

The scope of the present invention is defined solely by the appended claims, and is not affected to any degree by the statements within this summary.

A first measuring device for measuring an analyte extracted through skin of a living body embodying features of the present invention includes a noninvasive sampling unit for extracting the analyte from the living body through analyte transmission paths formed in an extracting region of the skin; an analyte detection unit for detecting a quantity of extracted analyte; and a control unit for obtaining a corrected analyte quantity based on the quantity of the extracted analyte and number of analyte transmission paths in the extracting region.

A second measuring device for measuring an analyte extracted through skin of a living body embodying features of the present invention includes: a noninvasive sampling unit for extracting the analyte from the living body through analyte transmission paths formed in an extracting region of the skin; an analyte detecting unit for detecting a quantity of extracted analyte; and a control unit for obtaining a corrected analyte quantity based on the quantity of the extracted analyte and an aperture area of analyte transmission paths in the extracting region.

A third measuring device for measuring an analyte extracted through skin of a living body embodying features of the present invention includes: a noninvasive sampling unit for extracting the analyte from the living body through analyte transmission paths formed in an extracting region of the skin, the noninvasive sampling unit including an extracting electrode and a holding medium for holding extracted analyte by a current flowing to the extracting electrode; an analyte detecting unit for detecting a quantity of the extracted analyte; and a control unit for obtaining a corrected analyte quantity based on the quantity of the extracted analyte and a current flow result.

A fourth measuring device for measuring an analyte extracted through skin of a living body embodying features of the present invention includes: a sampling unit for noninvasively extracting the analyte by applying electrical energy to the skin; a monitor unit for monitoring applied electrical energy; a detection unit for detecting a quantity of extracted analyte; and a control unit for obtaining a corrected analyte quantity based on the quantity of the extracted analyte and a monitor result by the monitor unit.

A first measuring system for measuring an analyte extracted through skin of a living body embodying features of the present invention includes: noninvasive sampling means for extracting the analyte from the living body through analyte transmission paths formed in an extracting region of the skin; analyte detecting means for detecting a quantity of extracted analyte; and control means for obtaining a corrected analyte quantity based on the quantity of the extracted analyte and number of analyte transmission paths in the extracting region.

A second measuring system for measuring an analyte extracted through skin of a living body embodying features of the present invention includes: noninvasive sampling means for extracting the analyte from the living body through analyte transmission paths formed in an extracting region of the skin; analyte detecting means for detecting a quantity of extracted analyte; and control means for obtaining a corrected analyte quantity based on the quantity of the extracted analyte and an aperture area of analyte transmission paths in the extracting region.

A first method for measuring an analyte extracted through skin of a living body embodying features of the present invention includes: noninvasively extracting the analyte through analyte transmission paths formed in an extracting region of the skin; detecting an extracted analyte quantity; and obtaining a corrected analyte quantity based on the extracted analyte quantity and a number of analyte transmission paths formed in the extracting region.

A second method for measuring an analyte extracted through skin of a living body embodying features of the present invention includes: noninvasively extracting the analyte through analyte transmission paths formed in an extracting region of the skin; detecting an extracted analyte quantity; and obtaining a corrected analyte quantity based on the extracted analyte quantity and an aperture area of the analyte transmission paths formed in the extracting region.

A third method for measuring an analyte extracted through skin of a living body embodying features of the present invention includes: noninvasively extracting the analyte by applying electrical energy to the skin; monitoring applied electrical energy; detecting an extracted analyte quantity; and obtaining a corrected analyte quantity based on the extracted analyte quantity and a monitoring result of the applied electrical energy.

### BRIEF DESCRIPTION OF THE DRAWINGS

FIG. 1 shows an electrical circuit when an electrode is disposed and a current flows to an extraction region on the skin.

FIG. 2 shows an electrical circuit of the skin forming a transmission path.

FIG. 3 shows an electrical circuit of the skin forming a transmission path.

FIG. 4 shows a first measuring device embodying features of the present invention.

FIG. 5 shows a second measuring device embodying features of the present invention.

FIG. 6 shows an extraction unit of a measuring device embodying features of the present invention.

FIG. 7 is a graph showing the change over time of extracted glucose concentration and blood sugar level.

### DETAILED DESCRIPTION

In accordance with the present invention, an analyte present in a living body is noninvasively extracted through the skin. This method of extracting an analyte forms transmission paths for the analyte through the skin (e.g., macropores such as sweat glands and pores, and micropores such as the space between keratose cells) by administering a predetermined energy to the skin, and extracts the analyte through these paths.

In extraction methods that form analyte transmission paths in the skin using macropores and/or micropores so as to extract the analyte through the transmission paths, a problem arises inasmuch as it is difficult to obtain stable measurement of the analyte quantity due to fluctuations in the quantity of analyte extracted over time.

Examples of usable methods for determining the number of analyte transmission paths include methods for staining and visually counting extraction regions of the skin, methods for determining the number of analyte transmission paths by staining and photographing the extraction region of the skin and analyzing the obtained image, and methods for estimating the number of transmission paths from the results of a current flow, such as an electrical resistance value, when electric energy is applied to the extraction region of the skin. A method of estimating the number of transmission paths from the results of a current flow is described below.

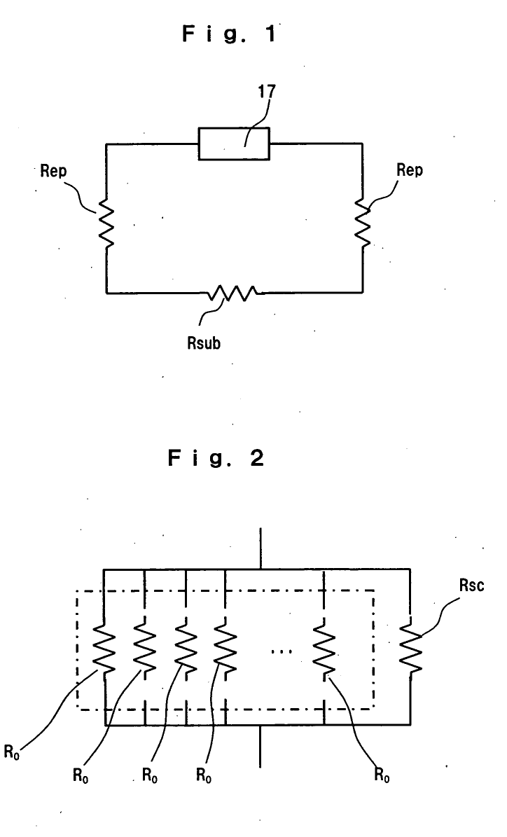

When electrodes are arranged in the extraction region of the skin to form the electrical circuit shown in FIG. 1, and the electrical resistance of the skin is designated Rep, and the in vivo electrical resistance is designated Rsub, then the combined electrical resistance R when a current flows can be represented by equation (1) below:
R=2×Rep+Rsub (1)

Although a horny layer, a granular layer, a spinous layer, and a basal layer are present in the structural tissue of the skin, since the electrical resistance of the horny layer is extremely large compared to the electrical resistance of the other layers, the condition of the horny layer may be deemed appropriate when considering the electrical resistance of the skin. The condition of the skin during extraction of the analyte is the condition wherein paths are formed in the horny layer which allow the transmission of the analyte. When the electrical resistance of each path is designated R₀, and, assuming all paths are identical, the resistance of the horny layer is designated Rsc, then the electrical circuit can be described as shown in FIG. 2. The resistance R₀ of each path is extremely small compared to the resistance Rsc of the horny layer. Therefore, the skin resistance Rep can be expressed by equation (2) below when the number of paths at a time t is designated N(t):

$${R}_{ep} = \frac{1}{\frac{1}{{R}_{o}} + \frac{1}{{R}_{o}} ⋯ + \frac{1}{{R}_{o}} + \frac{1}{{R}_{sc}}} = \frac{1}{\frac{N ( t )}{{R}_{o}} + \frac{1}{{R}_{sc}}} = \frac{{R}_{sc} {R}_{0}}{N ( t ) {R}_{sc} + {R}_{0}} \approx \frac{{R}_{0}}{N ( t )} \qquad ( 2 )$$

When the skin resistance at time t is designated R(t), equation (3) below can be derived from equations (1) and (2), and the number of analyte transmission paths can be estimated from the electrical resistance of the skin via equation (3):

$$R ( t ) = 2 \times Rep + Rsub \approx \frac{2 {R}_{0}}{N ( t )} + Rsub \qquad ( 3 )$$

When it is assumed that the analyte is extracted from the transmission path, the quantity of glucose extracted from each path is proportional to the blood sugar level, the quantity of glucose extracted from each path is equal, and the analyte transmission quantity per path is designated G(t) , then, the total analyte quantity gₜₒₜₐₗ(t) can be determined by equation (4) below:
gₜₒₜₐₗ(t)=G(t)N(t) (4)

The analyte transmission quantity G(t) per path can be determined by equation (5) below, which is derived from equations (3) and (4):
G(t)=gₜₒₜₐₗ(t)/N(t) (5)

When calibration is performed at time to and the relationship between G(t₀) and the actual blood sugar level is ascertained, the blood sugar level can be calculated at time t if the rate of change in G(t) from that time is known. When the rate of change is designated α, then α can be calculated using equations (3) and (5), as shown below:

$$α = \frac{G ( t )}{G ( {t}_{0} )} = \frac{{g}_{total} ( t ) / N ( t )}{{g}_{total} ( {t}_{0} ) / N ( {t}_{0} )} = \frac{( R ( t ) - {R}_{sub} ) \times {g}_{total} ( t )}{( R ( {t}_{0} ) - {R}_{sub} ) \times {g}_{total} ( {t}_{0} )} \qquad ( 6 )$$

Furthermore, when a constant-current power supply is used as the power source, equation (6) can be expressed as shown in equation (7) below because V(t)=R(t)×(current value), V(t₀)=R(t₀)×(current value), and Vsub=Rsub×(current value):

$$α = \frac{( V ( t ) - {V}_{sub} ) \times {g}_{total} ( t )}{( V ( {t}_{0} ) - {V}_{sub} ) \times {g}_{total} ( {t}_{0} )} \qquad ( 7 )$$

Although a method for estimating the number of analyte transmission paths at a measurement time t has been described above, the measurement time t may change according to the number of measurements. Furthermore, when a constant-voltage power supply is used as the power source, a can be determined from equation (6) using I(t)=(voltage value)/R(t), I(t₀)=(voltage value)/R(t₀), and Isub=(voltage value)/Rsub.

The analyte transmission path area is the total sum of the aperture areas of the transmission paths formed in the extraction region of the skin when extracting the analyte. Examples of methods for determining the aperture area of the analyte transmission paths include methods for determining the aperture area of the transmission paths by staining and photographing the extraction region of the skin and analyzing the obtained image, or methods for estimating the aperture area of the transmission paths from the results of a current flow, such as an electrical resistance value when electric energy is applied to the extraction region of the skin. A method of estimating the aperture area of transmission paths from electrical resistance values is described below.

As described above, when the electrical resistance of the skin is designated Rep, and the in vivo electrical resistance is designated Rsub, then the combined electrical resistance R when a current flows can be represented by equation (1).

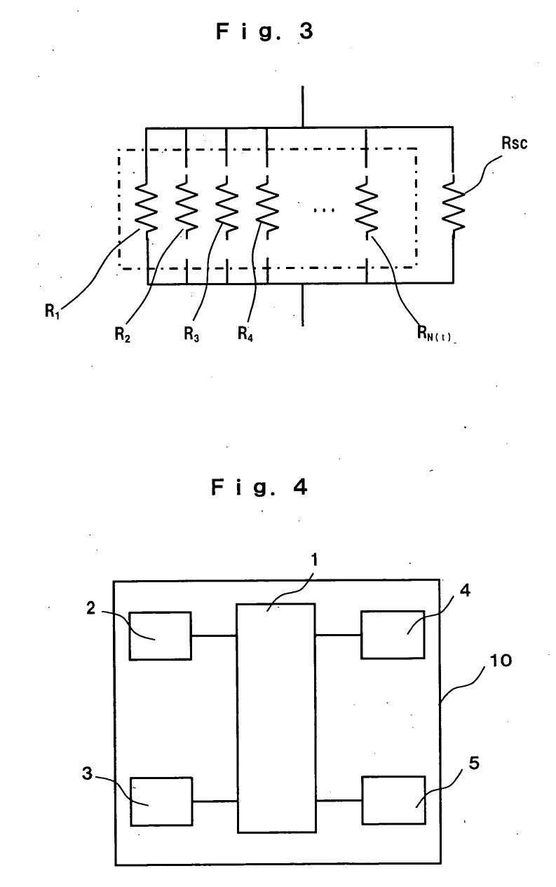

Since the number of paths N(t) changes with time and the aperture area of the respective apertures is not constant, the resistance value Ri of each path is different. When the resistance of the horny layer is designated Rsc, then, in the electrical circuit shown in FIG. 3, the Rep can be expressed by equation (8) below:

$${R}_{ep} = \frac{1}{( \frac{1}{{R}_{1}} + \frac{1}{{R}_{2}} ⋯ + \frac{1}{{R}_{N ( t )}} ) + \frac{1}{{R}_{sc}}} \qquad ( 8 )$$

When the target region is small and differences cannot be observed in the horny layer, the resistance value Ri of each path is inversely proportional to the respective aperture area Ai, and can be expressed by equation (9) below (where l is a proportionality constant)
Ri=k/Ai (9)

When the combined resistance of all paths of equation (8) is determined using equation (9), equation (10) is derived:

$$\frac{1}{{R}_{1}} + \frac{1}{{R}_{2}} ⋯ + \frac{1}{{R}_{N ( t )}} = \frac{{A}_{1}}{k} + \frac{{A}_{2}}{k} ⋯ + \frac{{A}_{N ( t )}}{k} = \frac{Σ Ai}{k} \qquad ( 10 )$$

When equation (10) is substituted in equation (8), equation (11) can be derived because the horny layer resistance Rsc is very much greater than the combined resistance of all the paths:

$${R}_{ep} = \frac{1}{\frac{Σ Ai}{k} + \frac{1}{{R}_{sc}}} \approx \frac{k}{A ( t )} \qquad ( 11 )$$

In equation (11), A(t) is the sum of the aperture areas of all paths at an optional measurement time t. When the skin resistance value at a specific time t is designated R(t), then equation (12) can be derived from equations (1) and (11), and the sum of the aperture areas of all paths can be estimated from the electrical resistance value of the skin using equation (12):
R(t)=2×Rep+Rsub≈(2k/A(t))+Rsub∴A(t)=2k/(R(t)−Rsub) (12)

When the analyte is glucose, the transmitted glucose quantity is corrected using the estimated path total aperture area because the utility of the correction by the sum of the path aperture areas has been confirmed. When it is assumed that the glucose is extracted from the transmission path, the quantity of glucose extracted from each path is proportional to the blood sugar level, and the quantity of glucose extracted per unit aperture area is constant at the same blood sugar level regardless of the path, and when the glucose transmitted quantity per unit aperture area is designated G(t), then the relationship between the total extracted glucose quantity gₜₒₜₐₗ(t) and the skin resistance value can be determined by equation (13) below:
gₜₒₜₐₗ(t)=G(t)A(t) (13)

When calibration is performed at time t₀ and the relationship between G(t₀) and the actual blood sugar level is ascertained, the blood sugar level can be calculated at time t if the rate of change in G(t) from that time is known. When the rate of change is designated α, then α can be calculated using equation (14), as shown below:

$$α = \frac{G ( t )}{G ( {t}_{0} )} = \frac{{g}_{total} ( t ) / A ( t )}{{g}_{total} ( {t}_{0} ) / A ( {t}_{0} )} = \frac{( R ( t ) - {R}_{sub} ) \times {g}_{total} ( t )}{( R ( {t}_{0} ) - {R}_{sub} ) \times {g}_{total} ( {t}_{0} )} \qquad ( 14 )$$

It is possible to perform correction from the ratio of the resistance value to the extracted glucose quantity at the time of measurement via equation (14). Furthermore, when the proportionality constant k is determined, the direct G(t) can be calculated since the relational equation of (15) below is obtained from equations (12) and (13):

$$G ( t ) = \frac{{g}_{total} ( t )}{A ( t )} = \frac{{kg}_{total} ( t )}{R ( t ) - {R}_{sub}} \qquad ( 15 )$$

Furthermore, when a constant-current power supply is used as the power source, equation (14) can be expressed as shown in equation (16) below because V(t)=R(t)×(current value), V(t₀)=R(t₀)×(current value), and Vsub=Rsub×(current value):

$$α = \frac{( V ( t ) - {V}_{sub} ) \times {g}_{total} ( t )}{( V ( {t}_{0} ) - {V}_{sub} ) \times {g}_{total} ( {t}_{0} )} \qquad ( 16 )$$

Although a method for estimating the surfaces area of analyte transmission paths at a measurement time t has been described above, the measurement time t may change according to the number of measurements. Furthermore, when a constant-voltage power supply is used as the power source, α can be determined from equation (14) using I(t)=(voltage value)/R(t) , I(t₀)=(voltage value)/R(t₀) , and Isub=(voltage value)/Rsub.

FIG. 4 is a schematic diagram showing an example of a control circuit when the percutaneous analyte measurement system is realized as a percutaneous analyte measuring device. The measuring device 10 of FIG. 4 is provided with a controller 1 for correction control of the analyte quantity and operational control of other structural elements, current result sensor 2 having a negative electrode and a positive electrode which are configured for placement on the skin of a subject and a voltmeter and power supply for supplying a voltage to these electrodes, an extractor 3 for extracting analyte in an extraction region of the skin of a subject, a measuring unit (sensor) 4 for measuring an analyte extracted by the extractor 3 and outputting a signal corresponding to the analyte quantity, and an output unit 5 for outputting the measurement result to a display. The measuring device 10 stores operation programs and analyte quantity correction process programs in memory, and stores data input from the current result sensor 2 and measuring unit 4 in memory.

The measuring device may be constructed with the controller 1, current result sensor 2, extractor 3, measuring unit 4, and output unit 5 all integrally formed so as to be configured for placement on a subject, or part of the structure may be separate in forming a system. For example, the current result sensor 2, extractor 3, and measuring unit 4 may be integrally formed so as to be configured for placement on a subject, and the controller 1 and output unit 5 may be separate structures. In this case, a personal computer may be used as the controller 1, and a personal computer display may be used as the output device 5. Furthermore, the current result sensor 2 and the extractor 3 may be integrally formed so as to be configured for placement on the subject, and the measuring unit 4, controller 1, and output unit 5 may be separate structures. Although the measuring device has a current result sensor 2 for estimating the number of analyte transmission paths or the aperture area of the analyte transmission paths from an electrical current result, such as the resistance value of the skin or the like, the current result sensor 2 may be omitted when the number of paths or path aperture area is determined by another method. While it is desirable that the measuring device detects the resistance value of the skin via the current result sensor 2, the voltage value may be detected when a constant-current power source is used as the power supply, and the current value also may be detected when a constant-voltage power source is used as the power supply. Furthermore, the power supply and voltmeter of the current result sensor 2 may be separate structures from the current result sensor 2, or may be integrally formed as a separate structural unit.

An example of a method for correcting the measured analyte quantity is described below. First, the device is installed on the subject, and the elapse of a predetermined wait time t₀ is awaited after starting extraction by the extractor 3 and detection of the current result (e.g., resistance value) by the current result sensor 2. This wait time is necessary because immediately after starting measurement, the analyte present in the transmission paths and on the surface of the skin is extracted, making the measurement inaccurate. The wait time to may be set to a suitable value depending on the condition of the skin of the subject. However, for simplicity, it is desirable that the wait time is set in the range of 0-30 minutes, with a setting in the range of 5-20 minutes being presently preferred. When measurement starts after performing a prior process to eliminate analyte present in the transmission paths and on the skin of the subject, the wait time to may be 0 minutes.

After the wait time to has elapsed, analyte is extracted in a predetermined time and the extracted analyte quantity g1 is measured once. The average resistance value R1 is determined for the resistance values detected during this predetermined time. The number of transmission paths N1 is determined from the average resistance value R1 obtained by the first measurement using equation (3). Furthermore, the analyte transmission quantity G1 per transmission path is determined from the analyte quantity g1, number of transmission paths N1, and equation (5). Moreover, it is desirable that calibration is performed relative to the measurement value g1 of the first analyte quantity, so as to calibrate the relationship between the measured value and an analyte quantity actually measured from blood. The calibration result relative to the analyte quantity g1 is designated C1.

After the first measurement, analyte is extracted for a predetermined time, and the analyte quantity g2 extracted at this time is measured a second time. The average resistance value R2 is determined for the resistance values detected during this predetermined time. A rate of change α1 is determined using equation (6), the analyte quantity g1, the average resistance value R1 of the first measurement, the analyte quantity g2, and the resistance value R2 of the second measurement. The in vivo resistance value Rsub may be an actual measured value, may be set beforehand from statistically obtained data, or may be a predicted value based on resistance measurement data. The predicted value can be determined as described below. For example, when using a constant-current power supply to supply a current, the resistance value (=(voltage value)/(current value)) decreases with the passage of the current application time, and stabilizes thereafter. The stabilized resistance value can be designated the predicted value of Rsub. This Rsub predicted value can be calculated from the amount of change in the voltage value or the resistance value when a current flows for a predetermined time. The corrected analyte quantity of the analyte quantity g2 can be determined from the analyte quantity g1 and the rate of change α1, and the corrected calibration result C2 of the analyte quantity g2 can be determined from C1 and the rate of change α1. In this case, the number of transmission paths N1 of the first measurement is set as the standard value of transmission paths, and the analyte transmission quantity G1 per transmission path is set as the standard value of analyte transmission quantity per unit path.

Thereafter, and in a similar manner for a predetermined number of times x (x=2, 3, . . . , x), the analyte quantity gx and average resistance value Rx are measured for each predetermined period, and the analyte quantity standard value per unit path and analyte transmission quantity Gx per unit path estimated from the average resistance value Rx and analyte quantity gx are used to obtain the corrected calibration result and corrected analyte quantity for the analyte quantity gx. Measurement ends when measurement has been performed x times.

Since the analyte quantity is normally corrected for the same number of transmission paths by this correction process, the analyte quantity changes as the number of transmission paths fluctuates, thereby eliminating the problem of no correlation with the actual in vivo analyte quantity.

Although the method described above is for correcting the analyte quantity gx (gₜₒₜₐₗ) using the analyte quantity per unit path and the standard value of the analyte Quantity per unit path, the present invention is not limited to this method. For example, if the correction item is an item other than the fluctuation of the number of transmission paths, the correction of the measured analyte quantity may be accomplished by this correction item rather than the correction of the fluctuation in the number of transmission paths. Furthermore, the analyte quantity gx (gₜₒₜₐₗ) also may be corrected using the ratio of the number of transmission paths Nx and the standard value N1 of the number of transmission paths. In this case, although the number of transmission paths is determined from the average resistance value within a predetermined time, the number of transmission paths also may be determined from the resistance value after the passage of a predetermined time. Furthermore, although the number of transmission paths and the analyte quantity per unit path in a first measurement were set as the standard values, the number of transmission paths and analyte quantity per unit path determined in a predetermined optional measurement may be set as the standard values. Although the analyte quantity is measured and corrected as described above, the analyte concentration may also be measured and corrected. The analyte quantity per unit path and the standard value of the analyte quantity per unit path are compared and their ratio is used in the correction of the analyte quantity as described above. However, the difference between these two values may also be used for correction. The measurements of the analyte quantity each time may be consecutive or intermittent.

Another example of a method for correcting the measured analyte quantity is described below.

After the previously mentioned wait time has elapsed, analyte is extracted for a predetermined time, and during that time the extracted analyte quantity g1 is measured a first time. The average resistance value R1 is determined for the resistance values detected during the predetermined time. The transmission path aperture area A1 (=2k/(R1−Rsub) is determined from equation (12) and the average resistance value R1 obtained from the first measurement. The analyte transmission quantity G1 (=g1(R1−Rsub)/2k) per unit area is determined from equation (13), the analyte quantity g1, and the transmission path aperture area A1. It is desirable that calibration is performed relative to the measurement value g1 of the first analyte quantity, so as to calibrate the relationship between the measured value and an analyte quantity actually measured from blood. The calibration result relative to the analyte quantity g1 is designated C1.

After the first measurement, analyte is extracted for a predetermined time, and the analyte quantity g2 extracted at this time is measured a second time. The average resistance value R2 is determined for the resistance values detected during this predetermined time. A rate of change α1 is determined using equation (14), the analyte quantity g1, the average resistance value R1 of the first measurement, the analyte quantity g2, and the resistance value R2 of the second measurement. The in vivo resistance value Rsub may be an actually measured value, may be set beforehand from statistically obtained data, or may be a predicted value from resistance measurement data. The predicted value can be determined as described below. For example, when using a constant-current power supply to supply a current, the resistance value (=(voltage value)/(current value)) decreases with the passage of the current application time, and stabilizes thereafter. The stabilized resistance value can be designated the predicted value of Rsub. This Rsub predicted value can be calculated from the amount of change in the voltage value or the resistance value when a current flows for a predetermined time. The corrected analyte quantity of the analyte quantity g2 can be determined from the analyte quantity g1 and the rate of change α1, and the corrected calibration result C2 of the analyte quantity g2 can be determined from C1 and the rate of change α1. In this case, the transmission path aperture area A1 determined from the analyte quantity g1 and the average resistance R1 of the first measurement is set as the standard value of the transmission path aperture area, and the analyte transmission quantity G1 per unit area is set as the standard value of analyte transmission quantity per unit area.

Thereafter, and in a similar manner for a predetermined number of times x (x=2, 3, . . . , x), the analyte quantity gx and average resistance value Rx are measured for each predetermined period, and the analyte quantity standard value per unit area and analyte transmission quantity Gx per unit area estimated from the average resistance value Rx and analyte quantity gx are used to obtain the corrected calibration result and corrected analyte quantity for the analyte quantity gx. Measurement ends when measurement has been performed x times.

Since the analyte quantity is normally corrected for the same transmission path aperture area by this correction process, the analyte quantity changes as the transmission path aperture area fluctuates, thereby eliminating the problem of no correlation with the actual in vivo analyte quantity.

Although the method described above is for correcting the analyte quantity gx (gₜₒₜₐₗ) using the ratio of the analyte quantity per unit area and the standard value of the analyte quantity per unit area, the present invention is not limited to this method. For example, if the correction item is an item other than the aperture areas of the number of transmission paths, the correction of the measured analyte quantity may be accomplished by this correction item rather than the correction of the fluctuation in the aperture area of the transmission paths. Furthermore, the correction may also be accomplished using the transmission path aperture area and the standard value of the transmission path aperture area. In this case, although the transmission path aperture area is determined from the average resistance value within a predetermined time, the transmission path aperture area also may be determined from the resistance value after the passage of a predetermined time. Furthermore, although the transmission path aperture area and the analyte quantity per unit area determined in a first measurement were set as the standard values, the transmission path aperture area and analyte quantity per unit area determined in a predetermined optional measurement may be set as the standard values. Although the analyte quantity is measured and corrected as described above, the analyte concentration may also be measured and corrected. The analyte quantity per unit area and the standard value of the analyte quantity per unit area are compared and their ratio is used in the correction of the analyte quantity as described above. However, the difference between these two values may also be used for correction. The measurements of the analyte quantity each time may be consecutive or intermittent.

Since the extracted analyte quantity is output as the in vivo analyte quantity of the subject as described above, it may be correlated with the analyte quantity measured from the blood of the subject. According to the present invention, even when correlating both these values, correlation may be accomplished just between the initially measured analyte quantity and the analyte quantity measured from the blood of the subject.

Methods for extracting analyte using macropores such as sweat glands and pores, and micropores such as the space between keratose cells as paths to transmit analyte through the skin may be used as the noninvasive analyte extraction method used by the extractor in the present invention. Known examples of such extraction methods include reverse iontophoresis for extracting analytes from a living body by passing an electrical current between electrodes arranged in the extraction region of a subject, sonophoresis for extracting analyte from a living body by exposing the extraction region of the skin to ultrasonic waves to reduce the barrier function of the skin and promote passive diffusion, methods applying negative pressure and suctioning for the extraction of analyte from a living body by suctioning the extraction region of the skin under negative pressure, and chemical enhancer methods for administering an enhancer to promote percutaneous movement of analyte to the extraction region of the skin. Two or more of these methods may also be combined to increase the number of transmission paths and increase the quantity of analyte extracted.

Among these analyte extraction methods, the reverse iontophoresis method is presently desirable. In this case, a direct current power supply or a combination of a direct current power supply and an alternating current power supply may be used as the power source for analyte extraction. From the perspective of applying a constant extraction current between negative and positive electrodes, it is desirable that a constant-current power supply is used as the direct current power source. In the case of the reverse iontophoresis method, the electrodes and power supply used for the analyte extraction may also be used in common for the electrodes and power supply used for transmission path detection. The sonophoresis method, negative pressure suction method, chemical enhancer method and the like may be combined with the reverse iontophoresis method.

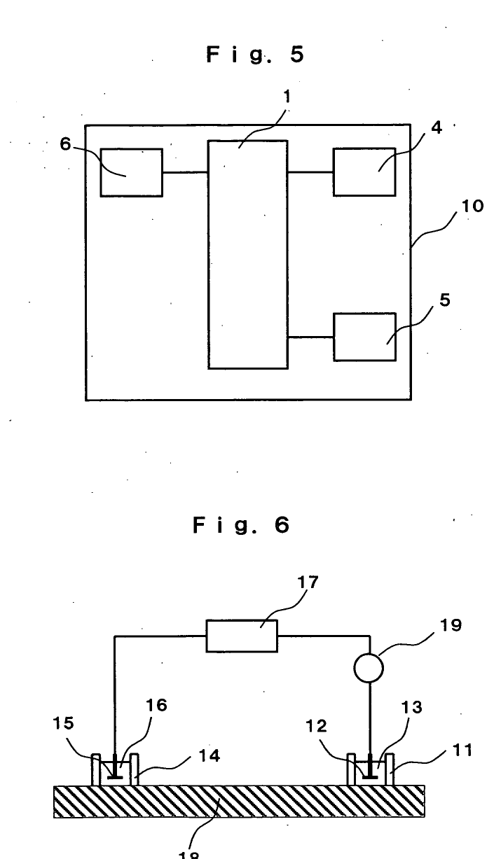

FIG. 5 is a schematic diagram showing the control circuit of a measuring device 10 for extracting analyte by the reverse iontophoresis method. Like parts in common with the measuring device of FIG. 5 are given like reference numbers. In the device of FIG. 5, the detection/extraction unit 6 extracts analyte by administering electrical energy, and is provided with at least a negative electrode and a positive electrode, a constant-current power supply for applying a voltage between these electrodes, and a voltmeter. These electrodes and power supply may also be used as the electrodes and power supply for transmission path detection (resistance detection).

In the measuring device of FIG. 5, the controller 1, detection/extraction unit 6, measuring unit 4, and output unit 5 may all be integrally formed so as to be configured for placement on a subject, or part of the structure may be separate in forming a system. For example, the detection/extraction unit 6 and measuring unit 4 may be integrally formed so as to be configured for placement on a subject. The controller 1 and output unit 5 may be separate structures. In this case, the measuring unit 4 may be integrated with the controller 1 and output device 5, or be a separate structure. The power supply or voltmeter of the detection/extraction unit 6 may be separate from the detection/extraction unit 6, and may be integrated with another structural part. Although the measuring device detects the resistance value of the skin via the detection/extraction unit 6, a voltage value may also be detected when a constant-current power supply is used as the power source.

FIG. 6 is a brief structural view of the detection/extraction unit 6 using a reverse iontophoresis method. In FIG. 6, a negative electrode chamber 11 and a positive electrode chamber 14 are placed on the skin 18 of a subject. An extraction medium 13 used for collecting extract and the negative electrode 12 are accommodated within the negative electrode chamber 11, and an extraction medium 16 and positive electrode 15 are accommodated within the positive electrode chamber 14. The negative electrode 12 and the positive electrode 15 are connected to a power supply 17, such that the voltage value can be monitored by a voltmeter 19. The power supply 17 is a constant-current power source. Furthermore, the extraction medium 13 and extraction medium 16 may be a solid, liquid, or semi solid (e.g., gel) material capable of collecting extract. Examples of useful extract media include purified water, ion-conductive aqueous solution, hydrogel, ion-conductive hydrogel, and the like. When a liquid is used as the extraction medium, absorbent porous material such as sponge, or hydrophilic polymer may be used.

When a voltage is applied by the power supply 17, the negative electrode 12 is negatively charged and the positive electrode 15 is positively charged. An analyte having a positive ionic charge is extracted into the extraction medium 16 on the positive electrode side. Although glucose is a noncharged material, it is mainly extracted into the extraction medium 13 on the negative electrode side.

Measurement of the analyte quantity each time may be consecutive or intermittent. At least the chamber performing extraction and measurement among the negative electrode chamber 11 and the positive electrode chamber 14 may be a disposable structure which is replaceable for each extraction/measurement. When the analyte quantity is intermittently measured, it is desirable that at least the chamber performing the analyte extraction/measurement is disposable.

Although a desirable analyte measured by the measurement device is glucose, other analytes include lactic acid, ascorbic acid, amino acid, enzyme substrate, pharmaceuticals, and the like. However, the analyte is not limited to these examples.

The use of glucose as an analyte is described below. Examples of usable methods for measuring glucose by the measuring unit (sensor) of the present invention include electrochemical detection methods used in high-performance liquid chromatography (HPLC), hexokinase methods (HK), glucose oxidase (GOD) electrode methods, glucose oxidase (GOD) colorimetry methods, and the like. The measuring unit using these methods may be disposable so as to be replaced for each measurement.

Correcting the measured glucose quantity in this way based on the number of transmission paths or transmission path aperture area provides close tracking of the fluctuation in the blood sugar level measured in collected blood, and the glucose quantity correlates with the blood sugar level.

Specific examples are described below.

### EXAMPLE

In FIG. 6, an 8 mmφ acrylic chamber is used as the negative electrode chamber 11 and the positive electrode chamber 14, ring-like AgCl was used as the negative electrode 12, and ring-like Ag was used as the positive electrode 15. Physiological saline solution was used as the extraction media 13 and 16, and a 0.2 mA constant-current power supply was used as the power source 17. Current application time for a single extraction was 15 minutes. The glucose quantity (concentration) collected in the physiological saline solution was measured using an electrochemical detection method and high-performance liquid chromatography (HPLC).

First, the skin of the subject was washed after fasting for 3 hours. Then the negative electrode chamber 11, positive electrode chamber 14, negative electrode 12, and positive electrode 14 are placed on the subject. Physiological saline solution was added to the chambers 11 and 14 as an extraction medium, and a 0.2 mA constant current was applied for 15 minutes. After the current was stopped, the physiological saline solution was removed from the negative electrode chamber 11 and the positive electrode chamber 14, and the chambers 11 and 14 and the electrodes 12 and 16 were washed.

(First Measurement)

Physiological saline solution was added to the chambers 11 and 14, and a 0.2 mA constant current was applied for 15 minutes. The voltage value was monitored by a voltmeter while the current was flowing, and the average electrical resistance value R1 during the current flow was determined. After the current was stopped, the physiological saline solution was collected from the chamber 11, and the concentration of the glucose extracted from the physiological saline solution was measured by HPLC. Thereafter, the chambers 11 and 14, and the electrodes 12 and 15 were washed.

(Second Through 24^{th} Measurements)

The second through 24^{th} measurement were repeated in the same sequence as in the first measurement. After the 8^{th} measurement, the subject ate and no further measurements were performed.

In the present embodiment, the voltage is monitored during the current flow, and the average electrical resistance value during the current flow is determined for each measurement period. The rate of change α was determined based on:

$$α = \frac{( R ( t ) - {R}_{sub} ) {g}_{total} ( t )}{( R ( {t}_{o} ) - {R}_{sub} ) \times {g}_{total} ( {t}_{o} )}$$

and the extracted glucose concentration was corrected using a personal computer in which the correction process program was installed. Specifically, the R(t₀) and gₜₒₜₐₗ(t₀) were determined from the first measurement value, and the rate of change α was determined from the measurement value of each measurement period. The in vivo resistance value Rsub of the subject was 4.19 kΩ. The data of the obtained measurement results are shown in Table 1, and the data of blood sugar levels measured from blood collected from the subject are shown in Table 2. In Table 1, the column “glucose concentration (uncorrected)” was calibrated for the first extracted glucose quantity, and the glucose extraction quantity was converted to concentration and correlated with the actual blood sugar level. The “extracted glucose concentration (corrected)” was calibrated for the first extracted glucose quantity, and the correction process of the present invention was performed for the calibration results of the second and subsequent extracted glucose quantities.

TABLE 1

| | | | Average | | | |
|---|---|---|---|---|---|---|
| | | | electrical | | Uncorrected | Corrected |
| | Elapsed | Average | resistance | Glucose | glucose | glucose |
| | time | voltage | value | concentration | concentration | concentration |
| | (minute) | (V) | (kΩ) | (ppb) | (mg/dl) | (mg/dl) |
| Wait | 15 | — | — | — | — | — |
| time | | | | | | |
| First | 31 | 1.72 | 8.6 | 41.44 | 82.5 | 82.5 |
| Second | 48 | 1.69 | 8.45 | 43.13 | 85.86 | 82.94 |
| Third | 64 | 1.6 | 8 | 48.78 | 97.11 | 83.9 |
| Fourth | 81 | 1.55 | 7.75 | 55.52 | 110.53 | 89.23 |
| Fifth | 98 | 1.52 | 7.6 | 59.53 | 118.52 | 91.64 |
| Sixth | 114 | 1.49 | 7.45 | 66.19 | 131.76 | 97.41 |
| Seventh | 130 | 1.47 | 7.35 | 67.61 | 134.6 | 96.45 |
| Eighth | 146 | 1.45 | 7.25 | 72.11 | 143.56 | 99.61 |
| Ninth | 163 | 1.44 | 7.2 | 73.09 | 145.5 | 99.32 |
| Tenth | 180 | 1.43 | 7.15 | 95.51 | 190.13 | 127.63 |
| 11th | 196 | 1.4 | 7 | 128.7 | 256.2 | 163.26 |
| 12th | 213 | 1.4 | 7 | 147.46 | 293.55 | 187.06 |
| 13th | 230 | 1.37 | 6.85 | 170.83 | 340.07 | 205.14 |
| 14th | 246 | 1.34 | 6.7 | 185.59 | 369.45 | 210.29 |
| 15th | 263 | 1.34 | 6.7 | 196.78 | 391.73 | 222.97 |
| 16th | 279 | 1.34 | 6.7 | 194.14 | 386.47 | 219.98 |
| 17th | 296 | 1.34 | 6.7 | 185.27 | 368.83 | 209.93 |
| 18th | 312 | 1.33 | 6.65 | 179.9 | 358.13 | 199.78 |
| 19th | 329 | 1.3 | 6.5 | 173.45 | 345.28 | 180.88 |
| 20th | 345 | 1.29 | 6.45 | 167.35 | 333.14 | 170.74 |
| 21th | 362 | 1.28 | 6.4 | 158.46 | 315.44 | 158.09 |
| 22th | 378 | 1.27 | 6.35 | 145.9 | 290.46 | 142.27 |
| 23th | 395 | 1.28 | 6.4 | 135.07 | 268.89 | 134.76 |
| 24th | 411 | 1.27 | 6.35 | 119.9 | 238.7 | 116.91 |

TABLE 2

| | | | | | | | | | |
|---|---|---|---|---|---|---|---|---|---|
| Elapsed | 3 | 117 | 150 | 166 | 183 | 199 | 216 | 232 | 249 |
| time | | | | | | | | | |
| (min- | | | | | | | | | |
| ute) | | | | | | | | | |
| Blood | 83 | 79 | 91 | 124 | 126 | 167 | 185 | 190 | 197 |
| sugar | | | | | | | | | |
| level | | | | | | | | | |
| (mg/dl) | | | | | | | | | |
| Elapsed | 265 | 281 | 298 | 315 | 331 | 348 | 364 | 381 | 397 |
| time | | | | | | | | | |
| (min- | | | | | | | | | |
| ute) | | | | | | | | | |
| Blood | 194 | 166 | 148 | 146 | 136 | 123 | 110 | 103 | 92 |
| sugar | | | | | | | | | |
| level | | | | | | | | | |
| (mg/dl) | | | | | | | | | |

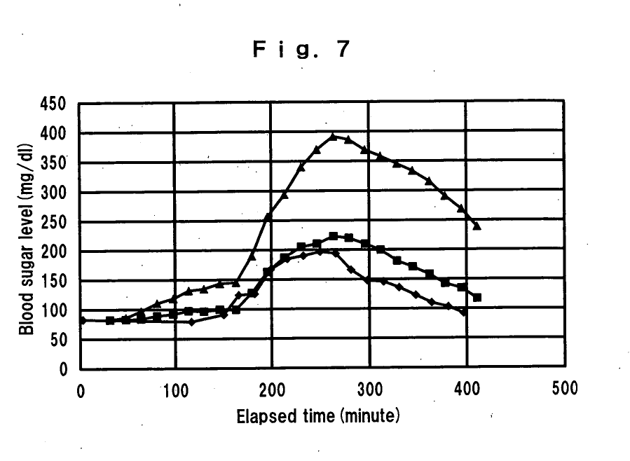

The measurement results are shown in FIG. 7. In FIG. 7, the (♦) symbol represents the blood sugar level measured from collected blood; the (▪) symbol represents the extracted glucose concentration after performing the correction process of the embodiment of the present invention; and the (▴) symbol represents extracted glucose concentration which was not subjected to the correction process.

In FIG. 7, the calibration was performed for the measurement point 1 (first measurement value), and the measured glucose extraction quantity was converted to concentration and correlated with the actual blood sugar level. Therefore, the measurement value and the actual blood sugar level were equal at measurement point 1. At measurement point 2 and thereafter, the fluctuation ((▴) in FIG. 7) of the extracted glucose concentration which was not corrected differs greatly from the fluctuation in the blood sugar level. However, the extracted glucose concentration (▪) in FIG. 7), which was corrected by the correction process of the embodiment of the present invention tracks the actual blood sugar level compared to uncorrected glucose concentration.

Although the present invention has been fully described by way of examples, it is to be noted that various changes and modifications will be apparent to those skilled in the art.

The foregoing detailed description and accompanying drawings have been provided by way of explanation and illustration, and are not intended to limit the scope of the appended claims. Many variations in the presently preferred embodiments illustrated herein will be obvious to one of ordinary skill in the art, and remain within the scope of the appended claims and their equivalents.

What is claimed is:

1. A measuring device for measuring an analyte extracted through skin of a living body, comprising:
a noninvasive sampling unit for extracting the analyte from the living body through analyte transmission paths formed in an extracting region of the skin;
an analyte detection unit for detecting a quantity of extracted analyte; and
a control unit for obtaining a corrected analyte quantity based on the quantity of the extracted analyte and number of analyte transmission paths in the extracting region.

2. The measuring device of claim 1, further comprising a plurality of electrodes comprising an extracting electrode, and a power supply for applying an electric current between the electrodes, wherein the control unit obtains the number of analyte transmission paths from a current result flowing between the electrodes.

3. The measuring device of claim 2, wherein the noninvasive sampling unit comprises the extracting electrode and a holding medium for holding the extracted analyte.

4. The measuring device of claim 2, wherein the current result comprises a resistance value, a voltage value, or a current value.

5. The measuring device of claim 1, wherein the control unit compares the number of analyte transmission paths to a standard value of number of transmission paths, and obtains the corrected analyte quantity based on a comparison result.

6. The measuring device of claim 5, wherein the comparison result comprises a ratio of the number of analyte transmission paths and the standard value of number of transmission paths.

7. The measuring device of claim 1, wherein the quantity of the extracted analyte comprises a concentration of the extracted analyte and the corrected analyte quantity comprises a corrected analyte concentration.

8. The measuring device of claim 1, wherein the control unit compares analyte quantity per unit number of transmission paths to a standard value of analyte quantity per unit number of transmission paths, and obtains the corrected analyte quantity based on a comparison result.

9. The measuring device of claim 8, wherein the comparison result comprises a ratio of the analyte quantity per unit number of transmission paths and the standard value of analyte quantity per unit number of transmission paths.

10. The measuring device of claim 1, wherein the analyte comprises glucose.

11. A measuring system for measuring an analyte extracted through skin of a living body, comprising:
noninvasive sampling means for extracting the analyte from the living body through analyte transmission paths formed in an extracting region of the skin;
analyte detecting means for detecting a quantity of extracted analyte; and
control means for obtaining a corrected analyte quantity based on the quantity of the extracted analyte and number of analyte transmission paths in the extracting region.

12. A measuring device for measuring an analyte extracted through skin of a living body, comprising:
a noninvasive sampling unit for extracting the analyte from the living body through analyte transmission paths formed in an extracting region of the skin;
an analyte detecting unit for detecting a quantity of extracted analyte; and
a control unit for obtaining a corrected analyte quantity based on the quantity of the extracted analyte and an aperture area of analyte transmission paths in the extracting region.

13. The measuring device of claim 12, further comprising a plurality of electrodes comprising an extracting electrode, and a power supply for applying an electric current between the electrodes, wherein the control unit obtains the aperture area of analyte transmission paths from a current result flowing between the electrodes.

14. The measuring device of claim 13, wherein the noninvasive sampling unit comprises the extracting electrode and a holding medium for holding the extracted analyte.

15. The measuring device of claim 13, wherein the current result comprises a resistance value, a voltage value, or a current value.

16. The measuring device of claim 12, wherein the control unit compares the aperture area of analyte transmission paths to a standard value of aperture area of analyte transmission paths, and obtains the corrected analyte quantity based on a comparison result.

17. The measuring device of claim 16, wherein the comparison result comprises a ratio of the aperture area of analyte transmission paths and the standard value of aperture area of analyte transmission paths.

18. The measuring device of claim 12, wherein the quantity of the extracted analyte comprises a concentration of the extracted analyte and the corrected analyte quantity comprises a corrected analyte concentration.

19. The measuring device of claim 12, wherein the control unit compares analyte quantity per unit aperture area of transmission paths to a standard value of analyte quantity per unit aperture area of transmission paths, and obtains the corrected analyte quantity based on a comparison result.

20. The measuring device of claim 19, wherein the comparison result comprises a ratio of the analyte quantity per unit aperture area and the standard value of analyte quantity per unit aperture area.

21. The measuring-device of claim 12, wherein the analyte comprises glucose.

22. A measuring system for measuring an analyte extracted through skin of a living body, comprising:
noninvasive sampling means for extracting the analyte from the living body through analyte transmission paths formed in an extracting region of the skin;
analyte detecting means for detecting a quantity of extracted analyte; and
control means for obtaining a corrected analyte quantity based on the quantity of the extracted analyte and an aperture area of analyte transmission paths in the extracting region.

23. A measuring device for measuring an analyte extracted through skin of a living body, comprising:
a noninvasive sampling unit for extracting the analyte from the living body through analyte transmission paths formed in an extracting region of the skin, the noninvasive sampling unit comprising an extracting electrode and a holding medium for holding extracted analyte by a current flowing to the extracting electrode;
an analyte detecting unit for detecting a quantity of the extracted analyte; and
a control unit for obtaining a corrected analyte quantity based on the quantity of the extracted analyte and a current flow result.

24. The measuring device of claim 23, wherein the quantity of the extracted analyte comprises a concentration of the extracted analyte and the corrected analyte quantity comprises a corrected analyte concentration.

25. The measuring device of claim 23, wherein the current flow result comprises a resistance value.

26. The measuring device of claim 25, wherein the control unit obtains the corrected analyte quantity based on the resistance value and a standard value of the resistance.

27. The measuring device of claim 26, wherein the control unit obtains the corrected analyte quantity based on a ratio of a difference between the resistance value and an in vivo resistance value, and a difference between the standard value of the resistance and the in vivo resistance value.

28. The measuring device of claim 23, wherein a constant current flows to the extracting electrode, and the current flow result comprises a voltage value.

29. The measuring device of claim 28, wherein the control unit obtains the corrected analyte quantity based on the voltage value and a standard voltage value.

30. The measuring device of claim 23, wherein a constant voltage flows to the extracting electrode, and the current flow result comprises a current value.

31. A measuring device for measuring an analyte extracted through skin of a living body, comprising:
a sampling unit for noninvasively extracting the analyte by applying electrical energy to the skin;
a monitor unit for monitoring applied electrical energy;
a detection unit for detecting a quantity of extracted analyte; and
a control unit for obtaining a corrected analyte quantity based on the quantity of the extracted analyte and a monitor result by the monitor unit.

32. The measuring device of claim 31, wherein the monitor result comprises a resistance value, a voltage value, or a current value.

33. The measuring device of claim 31, wherein the control unit obtains the corrected analyte quantity based on the monitor result and a standard value.

34. A method for measuring an analyte extracted through skin of a living body, comprising:
noninvasively extracting the analyte through analyte transmission paths formed in an extracting region of the skin;
detecting an extracted analyte quantity; and
obtaining a corrected analyte quantity based on the extracted analyte quantity and a number of analyte transmission paths formed in the extracting region.

35. The method of claim 34, wherein the analyte is extracted by applying electrical energy to the extracting region.

36. The method of claim 35, wherein the analyte comprises glucose.

37. A method for measuring an analyte extracted through skin of a living body, comprising:
noninvasively extracting the analyte through analyte transmission paths formed in an extracting region of the skin;
detecting an extracted analyte quantity; and
obtaining a corrected analyte quantity based on the extracted analyte quantity and an aperture area of the analyte transmission paths formed in the extracting region.

38. The method of claim 37, wherein the analyte is extracted by applying electrical energy to the extracting region.

39. The method of claim 37, wherein the analyte comprises glucose.

40. A method for measuring an analyte extracted through skin of a living body, comprising:
noninvasively extracting the analyte by applying electrical energy to the skin;
monitoring applied electrical energy;
detecting an extracted analyte quantity; and
obtaining a corrected analyte quantity based on the extracted analyte quantity and a monitoring result of the applied electrical energy.

41. The method of claim 40, wherein the monitoring result comprises a resistance value, a voltage value, or a current value.

42. The method of claim 40, wherein the corrected analyte quantity is obtained based on the monitoring result and a standard value.
